(19) United States
(12) Patent Application Publication
(10) Pub. No.: US 2025/0177370 A1
(43) Pub. Date: Jun. 5, 2025

(54) METHOD FOR ALLEVIATING UTERINE LEIOMYOMA USING NICOTINAMIDE

(71) Applicant: TAIPEI MEDICAL UNIVERSITY, Taipei City (TW)

(72) Inventors: Shih-Min HSIA, Taipei City (TW); Yi-Fen CHIANG, Taipei City (TW)

(21) Appl. No.: 18/526,010

(22) Filed: Dec. 1, 2023

(51) Int. Cl. A61K 31/455; A61P 35/00

(52) U.S. Cl. CPC A61K 31/455; A61P 35/00

(57) ABSTRACT

A method for alleviating uterine leiomyoma includes administering to a subject in need thereof a pharmaceutical composition which contains nicotinamide. The pharmaceutical composition further includes a pharmaceutically acceptable carrier. The pharmaceutical composition is administered by a route selected from the group consisting of oral administration, parenteral administration, and topical administration.

### FIELD

The present disclosure relates to a method for alleviating uterine leiomyoma using nicotinamide.

### BACKGROUND

Uterine leiomyoma, also known as uterine myoma or uterine fibroid, is a benign tumor caused by excessive cell proliferation and extracellular matrix (ECM) deposition. Factors such as diet, metabolism, and female hormones may contribute to the development of uterine leiomyoma. Common symptoms of uterine leiomyoma include uterine bleeding, pelvic pressure, pelvic pain, and reproductive dysfunction, which may even lead to infertility.

At present, no drug has been confirmed to effectively treat uterine leiomyoma. In clinical practice, the only viable approach for improving the symptoms of uterine leiomyoma is through surgical removal of the uterus.

Nicotinamide is a water-soluble form of vitamin B3 found in foods and used as dietary supplements and medications. It has been reported in Thompson B. C. et al. (2014), Exp. Dermatol., 23(7):509-511 that nicotinamide enhances DNA repair in primary melanocytes with ultraviolet (UV) radiation-induced DNA damage, and is considered to be useful in the prevention of skin cancer. In addition, it has been reported in Ekiguchi A. et al. (2013), Skin Aging New Research ebook Nova Science, 43-58 that the nicotinamide may reduce skin wrinkles, increase skin elasticity, and stimulate structural ECM proteins in dermal fibroblasts, thereby alleviating skin aging.

In spite of the aforesaid, there is still a need to develop an effective way for alleviating uterine leiomyoma.

### SUMMARY

Therefore, an object of the present disclosure is to provide a method for alleviating uterine leiomyoma, which can alleviate at least one of the drawbacks of the prior art, and which includes administering to a subject in need thereof a pharmaceutical composition containing nicotinamide.

### BRIEF DESCRIPTION OF THE DRAWINGS

Other features and advantages of the disclosure will become apparent in the following detailed description of the embodiment(s) with reference to the accompanying drawings. It is noted that various features may not be drawn to scale.

FIG. 1 shows the area density determined in each group of Example 1, infra, in which the symbol “***” represents p<0.001 (compared with the control group).

FIG. 2 shows the colony density determined in each group of Example 1, infra, in which the symbol “*” represents p<0.05 (compared with the control group).

FIG. 3 shows the relative expression level of fibronectin determined in each group of Example 2, infra, in which the symbol “***” represents p<0.001 (compared with the control group).

FIG. 4 shows the relative expression level of α-SMA determined in each group of Example 2, infra, in which the symbol “**” represents p<0.01 (compared with the control group).

FIG. 5 shows the relative expression level of COL1A1 determined in each group of Example 2, infra, in which the symbol “**” represents p<0.01 (compared with the control group).

### DETAILED DESCRIPTION

For the purpose of this specification, it will be clearly understood that the word “comprising” means “including but not limited to”, and that the word “comprises” has a corresponding meaning.

It is to be understood that, if any prior art publication is referred to herein, such reference does not constitute an admission that the publication forms a part of the common general knowledge in the art, in Taiwan or any other country.

Unless defined otherwise, all technical and scientific terms used herein have the meaning commonly understood by a person skilled in the art to which the present disclosure belongs. One skilled in the art will recognize many methods and materials similar or equivalent to those described herein, which could be used in the practice of the present disclosure. Indeed, the present disclosure is in no way limited to the methods and materials described.

The present disclosure provides a method for alleviating uterine leiomyoma, which includes administering to a subject in need thereof a pharmaceutical composition containing nicotinamide.

As used herein, the term “alleviating” or “alleviation” refers to at least partially reducing, ameliorating, relieving, controlling, treating or eliminating one or more clinical signs of a disease or disorder; and lowering, delaying, stopping or reversing the progression of severity regarding the condition or symptom being treated and preventing or decreasing the likelihood or probability thereof.

As used herein, the term “administration” or “administering” means introducing, providing or delivering a pre-determined active ingredient to a subject by any suitable routes to perform its intended function.

As used herein, the term “subject” refers to any animal of interest, such as humans, monkeys, cows, sheep, horses, pigs, goats, dogs, cats, mice, and rats. In certain embodiments, the subject is a human.

In certain embodiments, the subject suffers from the uterine leiomyoma.

According to the present disclosure, the nicotinamide suitable for use in this disclosure may be obtained as commercial products, or may be prepared using synthesis techniques well-known to those skilled in the art. In addition, the nicotinamide may be isolated and purified from a natural source using techniques well-known to those skilled in the art.

According to the present disclosure, the pharmaceutical composition may further include a pharmaceutically acceptable carrier widely employed in the art of drug-manufacturing. For instance, the pharmaceutically acceptable carrier may include one or more of the following agents: solvents, buffers, emulsifiers, suspending agents, decomposers, disintegrating agents, dispersing agents, binding agents, excipients, stabilizing agents, chelating agents, diluents, gelling agents, preservatives, wetting agents, lubricants, absorption delaying agents, liposomes, and the like. The choice and amount of the aforesaid agents are within the expertise and routine skills of those skilled in the art.

According to the present disclosure, the pharmaceutical composition may be formulated into a suitable dosage form for oral, parenteral or topical administration using technology well known to those skilled in the art.

According to the present disclosure, the dosage form suitable for oral administration includes, but is not limited to, sterile powders, tablets, troches, lozenges, pellets, capsules, dispersible powders or granules, solutions, suspensions, emulsions, syrup, elixir, slurry, and the like.

For parenteral administration, the pharmaceutical composition according to the present disclosure may be formulated into an injection, e.g., a sterile aqueous solution or a dispersion.

The pharmaceutical composition according to the present disclosure may be administered via one of the following parenteral routes: intraperitoneal injection, intramuscular injection, intravenous injection, intraarterial injection, intraepidermal injection, subcutaneous injection, intradermal injection, and intralesional injection. In certain embodiments, the pharmaceutical composition may be administered via intravenous injection.

According to the present disclosure, the pharmaceutical composition may be formulated into an external preparation suitable for topical application to the skin using technology well known to those skilled in the art. The external preparation includes, but is not limited to, emulsions, gels, ointments, creams, patches, liniments, powders, aerosols, sprays, lotions, serums, pastes, foams, drops, suspensions, salves, and bandages.

According to the present disclosure, the dose and frequency of administration of the pharmaceutical composition may vary depending on the following factors: the severity of the illness or disorder to be treated, routes of administration, and age, physical condition and response of the subject to be treated. In general, the pharmaceutical composition may be administered in a single dose or in several doses.

The disclosure will be further described by way of the following examples. However, it should be understood that the following examples are solely intended for the purpose of illustration and should not be construed as limiting the disclosure in practice.

### EXAMPLES

### General Experimental Materials:

### 1. Source and Cultivation of Eker Rat Uterine Leiomyoma Cell Line ELT-3

Eker rat uterine leiomyoma cell line ELT-3 was purchased from the American Type Culture Collection (ATCC, Manassas, Va., USA). The ELT-3 cells were grown in a 6 cm Petri dish containing Dulbecco's Modified Eagle's Medium (DMEM)/F12 [1:1 (v/v)] (Cassion, Cat. no. DFP29) supplemented with 10% fetal bovine serum (FBS, Corning Inc., Cat. no. 35-010-CV) and 1% antibiotic-antimycotic (Corning Inc., Cat. no. 30-004-CI). The ELT-3 cells were cultivated in an incubator at 37° C. with 5% CO₂. Medium change was performed every two to three days. Cell passage was performed when the cultured cells reached 80% to 90% of confluence.

### General Procedures:

### 1. Statistical Analysis

All the experiments described below were performed in triplicates. Statistical analysis was conducted using GraphPad Prism 8.0. The experimental data of all the test groups are expressed as mean±standard deviation (SD), and were analyzed using one-way analysis of variance (ANOVA) followed by Tukey's post hoc test, so as to evaluate the differences between the groups. Statistical significance is indicated by p<0.05.

### Example 1. Evaluation for the Effect of Nicotinamide on Inhibiting the Proliferation of Uterine Leiomyoma Cells

### Experimental Procedures:

### A. Crystal Violet Staining Assay

First, the ELT-3 cells prepared in section 1 of “General Experimental Materials” were divided into 3 groups, including a control group, and two experimental groups (i.e., experimental groups 1 and 2). Each group of the ELT-3 cells was seeded at a concentration of 1×10⁵ cells/well into a respective well of 6-well culture plates containing 2 mL of DMEM/F12 supplemented with 10% FBS and 1% penicillin-streptomycin, followed by cultivation in an incubator (37° C., 5% CO₂) for 24 hours.

Next, medium change was performed by adding a fresh culture medium supplemented with 1% FBS into the 6-well culture plate. Thereafter, each of the cell cultures of the experimental groups 1 and 2 was treated with a suitable amount of nicotinamide (Sigma-Aldrich, Cat. no. N0636) to reach final concentrations of 20 mM and 50 mM, respectively. In addition, the cell culture of the control group received no treatment. The treatment agent and the final concentration thereof for each group are summarized in Table 1 below.

TABLE 1

| Group | Treatment agent |
|---|---|
| Control group | — |
| Experimental group 1 | 20 mM nicotinamide |
| Experimental group 2 | 50 mM nicotinamide |

After cultivation in an incubator (37° C., 5% CO₂) for 48 hours, the culture medium in each well was removed, and the respective well was washed with phosphate-buffered saline (PBS) several times. Subsequently, 1 ml of methanol (ECHO Chemical Co. Ltd, Cat. no. MA1101) was added into the respective well to fix the cells for 10 minutes. Next, the liquid in each well was removed, followed by conducting staining using 5 mg/mL crystal violet (Sigma-Aldrich, Cat. no. C3886) for 15 minutes. Afterwards, the liquid in each well was removed, and the respective well was washed with double-distilled water (ddH₂O) three times. The resultant stained ELT-3 cells of each group were then observed under an optical microscope (Manufacturer: Olympus; Model no.: CKX41) at a magnification of 200×. One area of the respective stained ELT-3 cells was randomly selected and photographed, followed by subjecting the thus obtained images to determination of area density using an ImageJ software. Greater area density indicates a higher number of viable cells.

The data thus obtained were analyzed according to the procedures as described in section 1 of “General Procedures”.

### B. Colony Formation Assay

First, the ELT-3 cells prepared in section 1 of “General Experimental Materials” were divided into 3 groups, including a control group, and two experimental groups (i.e., experimental groups 1 and 2). Each group of the ELT-3 cells was seeded at a concentration of 5×10² cells/well into a respective well of 6-well culture plates containing 2 mL of DMEM/F12 supplemented with 10% FBS and 1% penicillin-streptomycin, followed by cultivation in an incubator (37° C., 5% CO₂) for 24 hours.

Next, medium change was performed by adding a fresh culture medium supplemented with 1% FBS into the 6-well culture plate. Thereafter, each of the cell cultures of the experimental groups 1 and 2 was treated with a suitable amount of nicotinamide to reach final concentrations of 20 mM and 50 mM, respectively. In addition, the cell culture of the control group received no treatment. The treatment agent and the final concentration thereof for each group are summarized in Table 1 above.

After cultivation in an incubator (37° C., 5% CO2) for 3 days, the culture medium of each group was removed to be replaced with a fresh culture medium supplemented with 10% FBS and 1% penicillin-streptomycin, followed by cultivation in an incubator (37° C., 5% CO₂) for 7 days to 10 days. Afterwards, the culture medium in each well was removed and the respective well was washed with phosphate-buffered saline (PBS) several times. Then, 95% methanol was added into the respective well to fix the thus formed cell colonies of each group for 10 minutes. Next, the liquid in each well was removed, followed by conducting staining using 0.5% crystal violet for 15 minutes. Afterwards, the liquid in each well was removed and the respective well was washed with double-distilled water (ddH₂O) three times. The resultant stained cell colonies of each group were then observed under the optical microscope at a magnification of 200×. One area of the respective stained cell colonies was randomly selected and photographed, followed by subjecting the thus obtained images to determination of colony density using the ImageJ software.

The data thus obtained were analyzed according to the procedures as described in section 1 of “General Procedures”.

### Results:

### A. Crystal Violet Staining Assay

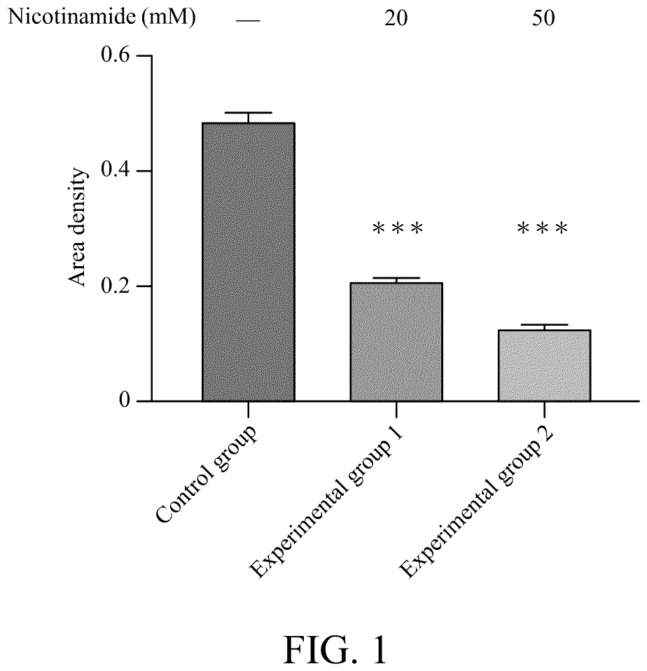

FIG. 1 shows the area density determined in each group. As shown in FIG. 1, compared with the control group, the area density determined in each of the experimental groups 1 and 2 exhibited a significant decrease, which became more prominent with increasing concentrations of nicotinamide.

### B. Colony Formation Assay

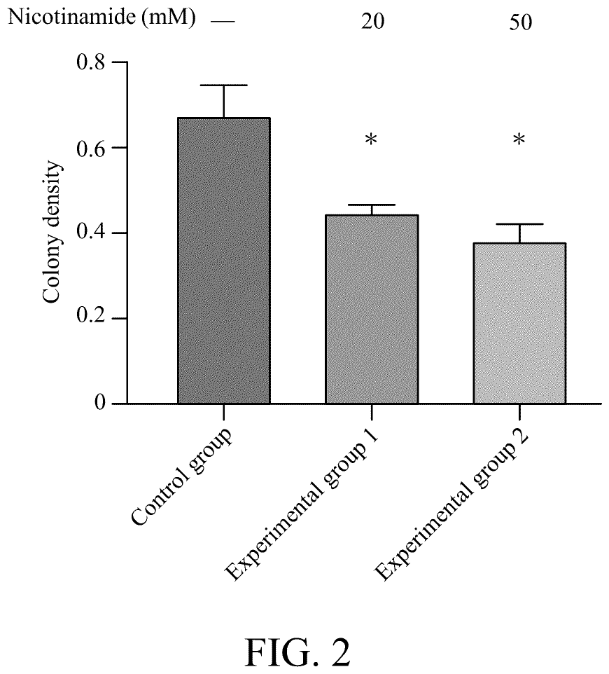

FIG. 2 shows the colony density determined in each group. As shown in FIG. 2, compared with the control group, the colony density determined in each of the experimental groups 1 and 2 exhibited a significant decrease, which became more prominent with increasing concentrations of nicotinamide.

Summarizing the above test results, it is clear that the nicotinamide is effective in inhibiting the proliferation of uterine leiomyoma cells.

### Example 2. Evaluation for the Effect of Nicotinamide on Inhibiting Generation of Extracellular Matrix (ECM) by Uterine Leiomyoma Cells

### Experimental Procedures:

First, the ELT-3 cells prepared in section 1 of “General Experimental Materials” were divided into 3 groups, including a control group, and two experimental groups (i.e., experimental groups 1 and 2). Each group of the ELT-3 cells was seeded at a concentration of 2×10⁵ cells/well into a 10-cm Petri dish containing 2 mL of DMEM/F12 supplemented with 10% FBS and 1% penicillin-streptomycin, followed by cultivation in an incubator (37° C., 5% CO₂) overnight.

Next, medium change was performed by adding a fresh culture medium supplemented with 1% FBS into the 10-cm Petri dish, followed by cultivation in an incubator (37° C., 5% CO₂) for 24 hours. Thereafter, each of the cell cultures of the experimental groups 1 and 2 was treated with a suitable amount of nicotinamide to reach final concentrations of 20 mM and 50 mM, respectively. In addition, the cell culture of the control group received no treatment. The treatment agent and the final concentration thereof for each group are summarized in Table 1 above.

After cultivation in an incubator (37° C., 5% CO₂) for 72 hours, each of the cell cultures was mixed with 100 μL of a RIPA lysis buffer (BIOMAN, Cat. no. 20121701) containing protease inhibitor cocktail (Roche, Cat. no. 4693159001). The resultant mixture was left to stand at 4° C. to allow a reaction to proceed for 30 minutes. After centrifugation at 12,000 rpm and 4° C. for 20 minutes, the supernatant thus obtained served as a total protein sample. The protein concentration in the total protein sample was determined using bicinchoninic acid (BCA) assay kit (T-Pro Biotechnology, Cat. no. JB04-D001) in accordance with the manufacturer's instructions.

The total protein sample of each group was subjected to sodium dodecyl sulfate-polyacrylamide gel electrophoresis (SDS-PAGE) analysis and Western Blotting analysis for detection of fibronectin, alpha-smooth muscle actin (α-SMA), and collagen type I alpha 1 chain (COL1A1) by virtue of the technique well known to and routinely used by one skilled in the art. In addition, glyceraldehyde-3-phosphate dehydrogenase (GAPDH) was used as an internal control.

The instruments and reagents used for SDS-PAGE analysis and Western Blotting analysis are as follows:
(1) SDS-PAGE analysis was performed using a Bio-Rad electrophoresis system (Bio-Rad Laboratories, Inc.);
(2) Protein transfer was performed using a Mini Trans-Blot® cell (Bio-Rad Laboratories, Inc.) and a polyvinylidene difluoride (PVDF) membrane;
(3) In Western Blotting analysis, primary and secondary antibodies used for detecting each protein are shown in Table 2 below; and
(4) Chemiluminescence staining was performed using ECL substrate kit (T-Pro Biotechnology, Cat. no. JT96-K004M), and signal detection was performed using luminescence imaging system (eBlot Photoelectric Technology, Cat. no. Touch Imager Mi).

TABLE 2

| Protein | Primary antibody | Secondary antibody |
|---|---|---|
| Fibronectin | Rabbit anti fibronectin | Goat anti rabbit IgG- |
| | polyclonal antibody (Abcam, | horseradish |
| | Cat. no. ab2413) | peroxidase (HRP) |
| α-SMA | Rabbit anti α-SMA polyclonal | antibody (Jackson |
| | antibody (Genetex, Cat. no. | ImmunoResearch |
| | GTX100034) | Laboratories, Cat. no. |
| COL1A1 | Rabbit anti COL1A1 polyclonal | 111-035-003) |
| | antibody (Genetex, Cat. no. | |
| | GTX112731) | |
| GAPDH | HRP-conjugated mouse anti | — |
| | GAPDH monoclonal antibody | |
| | (Proteintech, Cat. no. | |
| | HRP-60004) | |

Subsequently, ImageJ software was used for semi-quantitatively calculating the corresponding protein expression level. The expression level of the respective one of fibronectin, α-SMA, and COL1A1 in each group was normalized by the expression level of corresponding GAPDH thereof.

The relative expression level was calculated using the following Equation (1):

$$A = B / C \qquad ( 1 )$$

where A=relative expression level of fibronectin, α-SMA, or COL1A1
   B=normalized expression level of fibronectin, α-SMA, or COL1A1 determined in each group
   C=normalized expression level of fibronectin, α-SMA, or COL1A1 determined in the control group

The data thus obtained were analyzed according to the procedures as described in section 1 of “General Procedures”.

### Results:

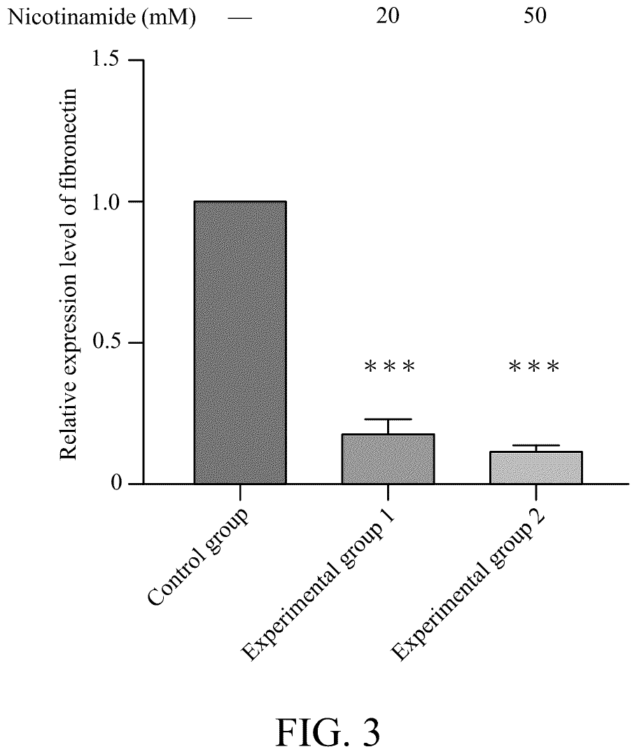

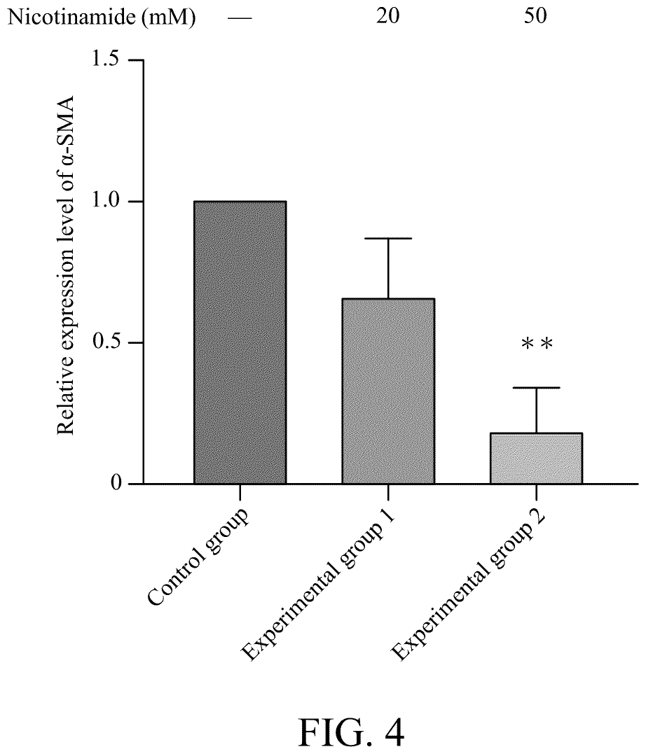

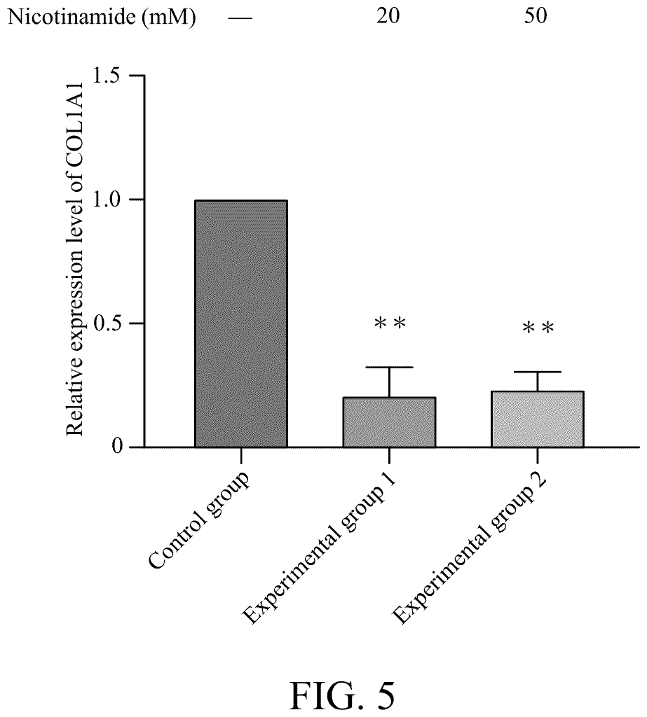

FIGS. 3 to 5 show the relative expression levels of fibronectin, α-SMA, and COL1A1 determined in each group, respectively. As shown in FIGS. 3 to 5, compared with the control group, the relative expression levels of fibronectin, α-SMA, and COL1A1 determined in each of the experimental groups 1 and 2 significantly decreased. In particular, the relative expression levels of fibronectin and α-SMA significantly decreased with increasing concentrations of nicotinamide. These results demonstrate that the nicotinamide can effectively inhibit the generation of ECM by uterine leiomyoma cells.

Summarizing the above test results, it is clear that the nicotinamide not only effectively inhibits the proliferation of uterine leiomyoma cells, but also significantly suppresses the generation of ECM by uterine leiomyoma cells. Therefore, the nicotinamide can prevent the occurrence and expansion of uterine leiomyoma, and is believed to have a high potential to be developed as a medicament for alleviating uterine leiomyoma.

In the description above, for the purposes of explanation, numerous specific details have been set forth in order to provide a thorough understanding of the embodiment(s). It will be apparent, however, to one skilled in the art, that one or more other embodiments may be practiced without some of these specific details. It should also be appreciated that reference throughout this specification to “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects; such does not mean that every one of these features needs to be practiced with the presence of all the other features. In other words, in any described embodiment, when implementation of one or more features or specific details does not affect implementation of another one or more features or specific details, said one or more features may be singled out and practiced alone without said another one or more features or specific details. It should be further noted that one or more features or specific details from one embodiment may be practiced together with one or more features or specific details from another embodiment, where appropriate, in the practice of the disclosure.

While the disclosure has been described in connection with what is (are) considered the exemplary embodiment(s), it is understood that this disclosure is not limited to the disclosed embodiment(s) but is intended to cover various arrangements included within the spirit and scope of the broadest interpretation so as to encompass all such modifications and equivalent arrangements.

What is claimed is:

1. A method for alleviating uterine leiomyoma, comprising administering to a subject in need thereof a pharmaceutical composition including nicotinamide.

2. The method as claimed in claim 1, wherein the pharmaceutical composition further includes a pharmaceutically acceptable carrier.

3. The method as claimed in claim 1, wherein the pharmaceutical composition is administered by a route selected from the group consisting of oral administration, parenteral administration, and topical administration.
